(19) United States
(12) Patent Application Publication
(10) Pub. No.: US 2021/0290603 A1
(43) Pub. Date: Sep. 23, 2021

(54) METHODS FOR TREATING ACNE

(71) Applicant: Lars Brichta, Brooklyn, NY (US)

(72) Inventor: Lars Brichta, Brooklyn, NY (US)

(73) Assignee: ChemistryRX, Folcroft, PA (US)

(21) Appl. No.: 17/249,917

(22) Filed: Mar. 18, 2021

Related U.S. Application Data
(60) Provisional application No. 62/991,168, filed on Mar. 18, 2020.

(51) Int. Cl. A61K 31/44; A61K 9/06; A61K 9/00; A61K 47/06

(52) U.S. Cl. CPC A61K 31/44; A61K 47/06; A61K 9/0014; A61K 9/06

(57) ABSTRACT

Compositions and methods for treating inflammation of the skin, acne, and acneiform disorders using a topical acetylcholinesterase inhibitor and a base are described herein.

### CROSS-REFERENCE TO RELATED APPLICATIONS

This application claims priority from U.S. Provisional No. 62/991,168, entitled “Methods for Treating Acne,” filed Mar. 18, 2020, the entirety of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Acne vulgaris is a common disease. Acne vulgaris most commonly occurs on oily areas of the skin with high sebaceous gland concentration. The areas of high sebaceous gland concentration are the face, ears, retroauricular areas (e.g. behind the ears), chest, back, and occasionally the neck and upper arms. Acneiform eruptions can occur wherever there is a pilosebaceous unit or sebaceous follicle which does include the entire surface of the skin.

The basic lesion in acne is the comedo commonly known as the blackhead. The comedo is created by retention of layers of dead skin known as keratin in the lining of the follicles. In addition to hyperkeratosis (which is thickening or retentive layering of keratin), there is an accumulation of sebum which is the lipid-laden product of the sebaceous gland. The cells of the sebaceous glands in which sebum originates are the sebocytes. The combination of the keratin and the sebum produces a plugging of the mouth or opening of the follicular canal, and papules are formed by inflammation around the comedones (plural of comedo). Depending upon the degree of inflammation, pustules, cysts, nodules, granulomatous reactions, scars, and keloids may develop.

### SUMMARY OF THE INVENTION

Not applicable

### DESCRIPTION OF THE DRAWINGS

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition, or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance, reduce, normalize, or adjust the growth, texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The terms “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is an adult or child human.

The term “treating” is used herein, for instance, in reference to methods of treating a disorder or a condition, and generally includes the administration of a compound or composition which reduces the frequency of, or delays the onset of, symptoms of a medical condition or enhance, reduce, normalize or adjust the growth, texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. For example, in the context of a bacterial, microbial, fungal, or protozoal infection, “treating” refers to the reduction in bacterial, microbial, fungal, or protozoal load and/or improvement in symptoms related to the infection.

As used herein, the term “therapeutic” means an agent utilized to treat, combat, ameliorate, prevent or improve an unwanted condition or disease of a subject. In part, embodiments described herein may be directed to the treatment of various skin diseases, conditions, or disorders or symptoms thereof, including, but not limited to, treat inflammation, acne, or acneiform disorders.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to topical compositions containing acetylcholinesterase inhibitors and a base, and methods for treating dermal inflammation, acne, and acneiform dermal disorders by administering such topical compositions to a subject in need of treatment. The topical compositions may reduce inflammation, symptoms relating to acne and acneiform dermal disorders, and other dermal disorders that create inflammation.

Acne, as used herein, encompasses various forms of acne vulgaris and other acneiform dermal disorders such as, for example, preadolescent acne, acne rosacea, premenstrual acne, acne venenata, acne cosmetica, pomade acne, acne detergicans, acne cosmetica, acne excoriee, gram negative acne, steroid acne, acne conglobata, nodulocystic acne, and the like and combinations thereof. Other types of acneiform dermal disorders that can be treated using the compositions of the invention include, for example, perioral dermatitis, seborrheic dermatitis in the presence of acne, gram negative folliculitis, sebaceous gland dysfunction, hidradenitis suppurativa, pseudofolliculitis barbae, folliculitis, and the like and combinations thereof.

Acetylcholinesterase inhibitors include any acetylcholinesterase inhibitor known in the art including, for example, donepezil, rivastigmine, galantamine, tacrine, metrifonate, huperzine-A, and the like and combinations thereof. Donepezil is described in U.S. Pat. No. 4,895,841, galantamine is described in U.S. Pat. No. 4,663,318, and rivastigmine is described in U.S. Pat. No. 4,948,807, each of which are hereby incorporated by reference in their entirety.

The dosage of acetylcholinesterase inhibitors encompassed by the invention may vary and may depend, for example, on the disease being treated or the severity of the disease, the weight, the physical condition, the health, the age of the subject. The acetylcholinesterase inhibitors can be provided in any amount capable of providing treatment. For example, the acetylcholinesterase inhibitor may be administered as single dose or divided doses of about 0.001 mg/kg body weight to about 750 mg/kg body weight, about 0.001 mg/kg body weight to about 500 mg/kg body weight, at least about 0.1 mg/kg body weight to about 300 mg/kg body weight or about 1 mg/kg body weight to about 100 mg/kg body weight, about 1 mg/kg body weight to about 50 mg/kg body weight or any range or individual dosage encompassed by these example ranges.

The concentration of acetylcholinesterase inhibitors in the compositions of embodiments can vary. For example, in some embodiments, the concentration of acetylcholinesterase inhibitors can be up to about 30% (w/w), and in some embodiments, the concentration of acetylcholinesterase inhibitors may be up to about 20% (w/w). In certain embodiments, the concentration of acetylcholinesterase inhibitors in the composition may be about 0.1% (w/w) to about 30% (w/w), about 0.25% (w/w) to about 20% (w/w), about 0.5% (w/w) to about 15% (w/w), about 1% (w/w) to about 15% (w/w), about 1% (w/w) to about 10% (w/w), or any range or individual concentration of acetylcholinesterase inhibitor encompassed by these example ranges. In particular embodiments, the composition may include about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of acetylcholinesterase inhibitor encompassed by these example ranges.

The compositions of various embodiments may include a base such as, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, and the like and combinations thereof, and in certain embodiments, the base may be an emollient base.

In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C₁₋₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygadalus amara (bitter almond) kernel oil, Vitis vinifera (Grape) seed extract, Triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

In some embodiments, the base may be cream base. Cream bases are semi-solid emulsions of oil and water. They are divided into two types: oil-in-water (O/W) creams which are composed of small droplets of oil dispersed in a continuous water phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturising as they provide an oily barrier which reduces water loss from the stratum corneum, the outermost layer of the skin. Cream bases typically include water, oil, emulsifier, and thickening agents, such as those discussed below.

In some embodiments, the base may be a moisturizing cream base. Moisturizing cream bases are composed of the same components as the cream bases described above with the addition of an emollient or humectant, that may provide a barrier that reduces water loss from the stratum corneum, the outermost layer of the skin. The emollient or humectant in a moisturizing cream base may be cetyl esters wax, stearyl alcohol, cetyl alcohol, and glycerin, or combinations thereof. Example cream bases and moisturizing cream bases include VersaBase (PCCA); Emollient cream, Vanishing cream, CeraVe, Vanicream, Vitamin E; Cliniderm; Dermabase (purified water, petrolatum, mineral oil, cetostearyl alcohol); Eucerin (water, petrolatum, mineral oil, ceresin, lanolin alcohol, methylchloroisothiozolinone, methylisothiazolinone); Glaxal (WellSpring Pharmaceutical Corp., Sarasota, Fla.); stearic acid cream, or any other pharmaceutical cream base used for topical formulations known to those skilled in the art.

In some embodiments, the base may be an ointment base. Ointments are compositions in which oil and water are provided in a ratio of from 7:1 to 2:1, from 5:1 to 3:1, or 4:1, and in some embodiments, the ointment may or may not include water, such as Aquaphor, Pracasil, and plasticized bases. Ointments are generally formulated using oils, waxes, water, alcohols, petroleum products, silicones, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

In some embodiments, the base may be an emollient base. Non-limiting examples of emollient bases includes C₉-C₁₄ linear or branched alkyl alcohols, C₃-C₁₄ linear or branched polyols, C₆-C₁₄ di-esters of C₆-C₁₂ diacids, hydrocarbons, natural waxes, vegetable oils, and silicones, branched chain esters, ethoxylated partial glyceride fatty acid esters, protein derivatives, lanolin and lanolin derivatives, and fatty alcohol ethoxylates, emollient oils, fatty acids, fatty alcohols and their esters. such as, for example, isononyl isonanoate, dioctyl sebacate, isooctyl isooctanoate, dioctyl adipate, squalane, petrolatum, mineral oil, carnauba wax, candelilla wax, beeswax, sunflower oil, sesame oil, olive oil, cyclomethicone and dimethicone.

In some embodiments, the emollient base may be or may include polyols having the formula:

HOCH₂—[CHOH]_{X}CH₂OH

wherein the index x is an integer from 1 to 20. In some embodiments, x is an integer from 1 to 10. Examples, of such polyols include glycerol, erythritol, xylitol, (2R,3R)-butane-1,2,3,4-tetrol, (2S,3R)-butane-1,2,3,4-tetraol, (2R,3S)-butane-1,2,3,4-tetraol, (2S,3S)-butane-1,2,3,4-tetrol, (2R,3R,4R)-pentane-1,2,3,4,5-pentol, (2S,3R,4R)-pentane-1,2,3,4,5-pentol, (2R,3S,4R)-pentane-1,2,3,4,5-pentol, (2R,3R,4S)-pentane-1,2,3,4,5-pentol, (2S,3S,4R)-pentane-1,2,3,4,5-pentol, (2S,3R,4S)-pentane-1,2,3,4,5-pentol, (2R,3S,4S)-pentane-1,2,3,4,5-pentol, and (2S,3S,4S)-pentane-1,2,3,4,5-pentol. In some embodiments, the emollient base may be glycerol.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 45% (w/w) to about 99.75% (w/w) of the total composition, or any range or individual concentration known in the art.

In some embodiments, the compositions may further include a solubility enhancer. A solubility enhancer may be necessary to produce a topical composition that effectively delivers the aminothiols, biguanides, and combinations thereof, to affected tissues.

The solubility enhancers are not limited and may include various known solubility enhancers and combinations thereof. In particular embodiments, the solubility enhancer may be, for example, ethyl acetate, ethanol, methanol, dimethylformamide (DMF), acetone, acetonitrile, tetrahydrofuran (THF), acetic acid, dimethyl sulfoxide (DMSO), chloroform, propylene glycol, polyethylene glycol, propane-1,3-dioland the like and combinations thereof, and in some embodiments, the solubility enhancer may be DMSO. The composition may include about 10% (w/w) to about 40% (w/w) solubility enhancer, and in some embodiments, the composition may include at least about 25% (w/w) to about 35% (w/w) solubility enhancer.

In some embodiments, the compositions described above may further include one or more pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

The compositions of various embodiments can be in any form, including, for example, liquid, creams, lotions, foams, liniments, and the like.

In some embodiments, the compositions described above may be formulated as a liquid. Liquid dosage forms for topical administration may include diluents such as, for example, alcohols, glycols, oils, water, and the like. Such compositions may also include wetting agents or emulsifiers. In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanoilin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

In some embodiments, the topical formulations can be in the form of a lotion. Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the topical formulations can be in the form of a foam. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

In some embodiments, the topical formulations can be in the form of a liniment. Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicilate, benzoin resin, or capsaicin.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

Vitamins include, for example, vitamin D, vitamin K, vitamin B (including niacinamide, nicotinic acid, C1-18 nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

In some embodiments, the compositions may further include a topical debriding agent such as, for example, papain/urea, balsam peru/castor oil/trypsin, chlorophyllin copper complex/papain/urea, collagenase, and the like and combinations thereof. The amount of the debriding agent in such compositions may be about 0.01% (w/w) to 5% (w/w) of the total composition.

In some embodiments, the compositions may further include a fragrant oil such as, for example, rosemary, lavender, vanilla, and the like and combinations thereof. The amount of the fragrant oil in such compositions may be about 0.01% (w/w) to 5% (w/w) of the total composition.

Other embodiments of the invention include methods for treating inflammation of the skin, acne, acneiform disorders, and the like and combinations thereof by administering any of the compositions described above. The methods of various embodiments may include the steps of administering a composition of the invention to the location of inflammation, acne, acneiform disorders, and the like and combinations thereof of a subject in need of treatment. The step of administering can be carried out by various means. For example, administering can be accomplished by applying the composition to the skin of a subject, and in some embodiments, the skin may be massaged or rubbed to facilitate contacting affected area. In some embodiments, the step of administering can be carried out one, two, three, four, or more times per day, and administering can be carried out the prescribed number of times per day for one week to six months or until the symptoms are resolved. In some embodiments, improvement in one or more symptoms may be observed within about 7 days of treatment, and in certain embodiments, improvement in one or more symptoms may be observed within about 1, about 2, about 3, about 4, about 5, or about 6 days after initial treatment.

A wide variety of methods may be used for preparing the formulations described above. The formulations may be prepared by combining together the components of the formulation, as described herein, at a temperature and for a time sufficient to provide a pharmaceutically acceptable composition. For example, in some embodiments, the compositions components of the compositions may be dissolved, suspended, dispersed or otherwise mixed in a selected carrier or vehicle, at an effective concentration such that the condition to be treated is relieved or ameliorated.

As is known in the art, certain means for administering may require the use of particular components of the formulation. Such components are described above and can be appropriately incorporated into the compositions.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

An acetylcholinesterase inhibitor containing cream will be prepared having the following ingredients:

TABLE 1
Cream compositions

| | Cream 1 |
|---|---|
| Donepezil HCl | 1% |
| Water | 30% |
| Emulsion stabilizer | 2% |
| Non-comedogenic cream | 67% |

### Example 2

Acetylcholinesterase inhibitor containing gels will be prepared having the following ingredients:

TABLE 2
Gel compositions

| | Gel 1 | Gel 2 |
|---|---|---|
| Donepezil HCl | 1% | 1% |
| Water | 30% | |
| Gransil RPS | 25% | |
| Gransil 314 | 44% | |
| Versabase Gel | | 99% |

1. A composition for treating inflammation of the skin comprising an acetylcholinesterase inhibitor and a base.

2. The composition of claim 1, wherein the amount of acetylcholinesterase inhibitor is about 0.5 wt. % to about 25 wt. % based on the total weight of the composition.

3. The composition of claim 1, wherein the acetylcholinesterase inhibitor is Donepezil.

4. The composition of claim 1, wherein the base is an emollient base, liposomal base, cream base, moisturizing cream base, or ointment base.

5. The composition of claim 1, wherein the base has a concentration of about 45% (w/w) to about 99.75% (w/w) of the total composition.

6. The composition of claim 1, further comprising a solubility enhancer selected from the group consisting of ethyl acetate, ethanol, methanol, dimethylformamide, acetone, acetonitrile, tetrahydrofuran, acetic acid, dimethyl sulfoxide, chloroform, propylene glycol, polyethylene glycol, propane-1,3-diol, and combinations thereof.

7. The composition of claim 8, wherein the concentration of solubility enhancer is about 10% (w/w) to about 40% (w/w).

8. The composition of claim 1, further comprising vitamins, antioxidants, emulsifying agents, humectants, analgesic agents, topical debriding agents, fragrant oils, and combinations thereof.

9. The composition of claim 1, further comprising pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and combinations thereof.

10. The composition of claim 1, wherein the composition is in the form of a liquid, cream, lotion, foam, or liniment.

11. A method of treating inflammation of the skin comprising:
administering a therapeutically effective amount of a composition comprising an acetylcholinesterase inhibitor and a base to a subject in need of treatment.

12. The method of claim 11, wherein the amount of acetylcholinesterase inhibitor is about 0.5 wt. % to about 25 wt. % based on the total weight of the composition.

13. The method of claim 11, wherein the acetylcholinesterase inhibitor is Donepezil.

14. The method of claim 11, wherein the base is an emollient base, liposomal base, cream base, moisturizing cream base, or ointment base.

15. The method of claim 11, wherein the base has a concentration of about 45% (w/w) to about 99.75% (w/w) of the total composition.

16. The method of claim 11, further comprising a solubility enhancer selected from the group consisting of ethyl acetate, ethanol, methanol, dimethylformamide, acetone, acetonitrile, tetrahydrofuran, acetic acid, dimethyl sulfoxide, chloroform, propylene glycol, polyethylene glycol, propane-1,3-diol, and combinations thereof.

17. The method of claim 16, wherein the concentration of solubility enhancer is about 10% (w/w) to about 40% (w/w).

18. The method of claim 11, further comprising vitamins, antioxidants, emulsifying agents, humectants, analgesic agents, topical debriding agents, fragrant oils, and combinations thereof.

19. The method of claim 11, further comprising pharmaceutically acceptable diluents, fillers, disintegrants, binders, lubricants, surfactants, hydrophobic vehicles, water soluble

20. The methods of claim 11, wherein the inflammation is a result of acne or acneiform disorders of the skin.
